(19) United States
(12) Patent Application Publication
(10) Pub. No.: US 2002/0114767 A1
(43) Pub. Date: Aug. 22, 2002

(54) Oral hygiene preparation for inhibiting halitosis

(76) Inventor: Gunnar Rolla, Oslo (NO)

Correspondence Address: WENDEROTH, LIND & PONACK, L.L.P., 2033 K STREET N. W., SUITE 800, WASHINGTON, DC, 20006-1021, US

(21) Appl. No.: 09/988,718

(22) Filed: Nov. 20, 2001

Related U.S. Application Data
(60) Provisional application No. 60/252,091, filed on Nov. 21, 2000.

(30) Foreign Application Priority Data
Nov. 21, 2000 (NO) 2000-5877

(51) Int. Cl. A61K 7/16

(52) U.S. Cl. 424/49

(57) ABSTRACT

An oral hygiene preparation for inhibiting halitosis, in the form of a tablet or a powder forming a mouthwash upon dissolution in water, which contains copper(ll)gluconate and a disintegrating agent selected from sodium and calcium bicarbonate.

This invention relates to an oral hygiene preparation for inhibiting halitosis, in the form of a tablet or a powder which upon dissolution in water forms a mouthwash. The mouthwash thus produced is effective against oral malodour

It is well established that oral malodour is caused by volatile sulfur compounds (VSC) produced by bacterial catabolization of proteins in the oral cavity, in particular in the crypts at the back of the tongue and in periodontal pockets. The VSC are mainly hydrogen sulfide and methyl mercaptan, which have a very unpleasant odour, even in low concentrations. The VSC are also known to be able to penetrate oral epithelium and damage cells in the underlying connective tissue and bone, a process which is believed to be an important aspect of the initiation and progression of periodontal disease.

It is furthermore known that water-soluble salts of certain metals, such as divalent cations of zinc, copper and tin, can inhibit oral malodour. The metals mentioned above have high affinity for sulfur and eliminate the VSC by forming insoluble sulfides with precursors of the volatile sulfur compounds, a reaction which inhibits further formation of the odiferous gases in the oral cavity.

It is also well known that water soluble, cationic, antibacterial agents such as the bis-biguanides and the quaternary ammonium compounds are able to inhibit oral malodour when used as mouth rinses.

Japanese Patent Application JP 1996/356310 (Publication JP 98182384 A2) discloses a composition for oral cavity, which can be used for prevention of bad breath. Possible components of the composition are triclosan, sodium bicarbonate, menthol and other fragrances.

Japanese Patent Application JP 1988/317621 (Publication JP 90164816 A2) relates to an aqueous drink containing ferrous sulfate and sodium bicarbonate. It is stated in the application that halitosis is inhibited by drinking said drink.

Japanese Patent Application JP 1985139538 (Publication JP 86197510 A2) relates to a composition which may be in the form of a toothpaste, tooth powder, mouthwash, gingiva massage cream or local liquid or a paste paint. The composition contains small amounts of nitroimidazole. The composition further contains extracts from leaves of camellia plant such as tea tree or camellia or sodium copper-chlorophyllin. The composition is used for prevention of periodental diseases and stomatosis and may also inhibit gram negative, anaerobic microorganisms from forming volatile sulfur compounds in the oral cavity.

WO 99/56714 relates to a bactericide for detergents which comprises an inorganic support having antifungal metal ions supported thereon, and a denture detergent containing the bactericide. The bactericide is specifically intended for dentures and it is stated that it suppresses bad breath and denture-specific stomatitis.

The article “Effects of new flavonoid gums eliminating bad breath”, Shokuhin Kogyo (SKGYAQ, 05598990); 1995; Vol.38 (4); pp. 70-8, relates to a chewing gum containing green tea flavonoids, chlorophyll copper complex and Hovenia dulcis. It is used for eliminating bad breath.

WO-0023040 relates to a dry composition for inhibiting oral malodour, which comprises:

a) at least one carrier material selected from saccharide materials (sweeteners);

b) at least one processing aid selected from oleaginous substances; and

c) at least one active ingredient selected among many different types, including metallic ions

Copper gluconate is mentioned as a source of metallic ions.

It has now been discovered that a mouthwash produced from a tablet or a powder, which can be rapidly solubilized in water and which contains divalent cations of copper, exhibits a marked inhibiting effect against oral malodour.

The present invention provides an oral hygiene preparation for inhibiting halitosis, in the form of a tablet or powder which upon dissolution in water forms a mouthwash. The preparation contains copper(ll)gluconate which provides cupric ions in water, and a physiologically acceptable disintegrating agent selected from sodium and calcium bicarbonate.

Copper(ll)gluconate has sufficient solubility so that all of it is dissolved when the composition is added to a suitable amount of water. Copper(ll)gluconate contains about 15% of copper. A suitable amount of copper in a tablet or a powder dosage is an amount which provides a copper concentration of 0.003% to 0.3% by weight in 10 ml of water, and preferably a concentration of about 0.015%. In order to obtain a suitable concentration of copper in the mouthwash, a tablet or a powder dosage should contain copper(ll)gluconate in an amount of 0.3-30 mg, preferably 0.5-5 mg, calculated as free cupric ions. This corresponds to 2-200 mg, preferably 3.5-35 mg, of copper gluconate.

The preparation according to the invention contains a physiologically acceptable disintegrating agent which facilitates the rapid solubilisation of the preparation, including the copper(ll)gluconate. The disintegrating agents are selected from sodium and calcium bicarbonate, which may be used in amounts of 20-2000 mg, in particular 50-500 mg in a tablet designed to be dissolved in 10 ml of water. In a powder the amount of disintegrating agent, such as sodium bicarbonate, will generally be less than in a tablet.

A particularly preferred preparation according to the invention is a tablet which contains 50-500 mg of sodium bicarbonate and 5-50 mg of copper(ll) gluconate.

The composition of the invention may also contain further components such as sweetening agents, flavouring agents, excipients and fillers. Examples of such other components are acesulfam K, aspartam, saccharin, xylitol and sorbitol.

Tablets in the form of lozenges containing copper are already on the market as dietary supplements or for inhibiting oral malodour. Such tablets are designed to be solubilized in the mouth by sucking and are then often only sparingly soluble in water. Such tablets are markedly different from the tablets described in the present application. An agent with an inhibiting effect on oral malodour is far more effective in the aqueous form of a mouthwash than in the dry state of a sucking tablet. The reason is that the VSC are highly soluble in water. The aqueous part of a mouthwash is accordingly effective as such, this being in addition to the effect of the solubilized agents present in the mouthwash, as mentioned above. This concept is supported by the well established observation that oral malodour is frequently associated with a dry mouth.

Thus, the present invention combines the availability and convenience in the use of tablets, powders or other solid compositions, with the superior effect exhibited by mouthwashes.

A further advantage of a mouthwash compared with a sucking tablet is that the use of a mouthwash involves a local effect only of the copper cations in the mouth, because the mouthwash is expectorated after use, whereas the ingredients in a sucking tablet would have to be swallowed.

### EXAMPLE 1

A powder designed to be dissolved in 20 ml of water:

| | |
|---|---|
| Cupric gluconate | 20 mg |
| Sodium bicarbonate | 50 mg |
| Xylitol | 2000 mg |

Sweeteners and flavours quantun satis

### EXAMPLE 2

Tablet designed to be dissolved in 10 ml of water:

| | |
|---|---|
| Cupric gluconate | 5 mg |
| Sodium bicarbonate | 100 mg |
| Xylitol/Sorbitol | 1000 mg |

Constituents ad 2000 mg

### EXAMPLE 3

Tablet designed to be dissolved in 10 ml of water:

| | |
|---|---|
| Cupric gluconate | 10 mg |
| Calcium bicarbonate | 90 mg |
| Acesulfam K quantum satis | |
| Constituents ad 500 mg | |

### EXAMPLE 4

Tablet designed to be dissolved in 20 ml of water:

| | |
|---|---|
| Cupric gluconate | 15 mg |
| Sodium bicarbonate | 200 mg |
| Xylitol | 2000 mg |

Constituents ad 3000 mg

1. A oral hygiene preparation for inhibiting halitosis, in the form of a tablet or a powder forming a mouthwash upon dissolution in water and containing copper(ll)gluconate, wherein said preparation also contains a physiologically acceptable disintegrating agent selected from sodium and calcium bicarbonate.

2. The oral hygiene preparation of claim 1, which contains a higher alcohol selected from xylitol and sorbitol.

3. The oral hygiene preparation of any of claims 1 and 2 in the form of a tablet or a powder dosage containing copper(ll)gluconate in an amount of 0.3-0.330 mg, calculated as free cupric ions, and said disintegrating agent in an amount of 20-2000 mg.

4. The oral hygiene preparation of claim 3, wherein the amount of copper(ll)gluconate, calculated as free cupric ions, is 0.5-5 mg.

5. The oral hygiene preparation of any of claims 3 and 4, wherein the amount of said disintegrating agent is 50-500 mg.

6. The oral hygiene preparation of claim I in the form of a tablet containing 50-500 mg of sodium bicarbonate and 5-50 mg of copper(ll)gluconate.
